(19) United States
(12) Patent Application Publication
(10) Pub. No.: US 2025/0082365 A1
(43) Pub. Date: Mar. 13, 2025

(54) METHOD AND DEVICE FOR ABRASION OF OUTER LAYER OF EPIDERMIS

(71) Applicant: MIRKA OY, Jeppo (FI)

(72) Inventors: Markus KASS, Bennäs (FI); Hans HEDE, Vörå (FI); Cecilia BJÖRKMAN, Nykarleby (FI); Teresa CZURYSZKIEWICZ, Karis (FI); Ann-Sofie FONSEN, Åbo (FI); Charlotta RISKU, Solf (FI); Johanna HAGMAN, Vasa (FI); Isabella NORRGÅRD, Vasa (FI)

(21) Appl. No.: 18/723,212

(22) PCT Filed: Dec. 20, 2022
(86) PCT No.: PCT/FI2022/050850

§ 371 Date: Jun. 21, 2024

(30) Foreign Application Priority Data
Dec. 23, 2021 (FI) 20216342

(51) Int. Cl. A61B 17/54; A61B 17/00

(52) U.S. Cl. CPC A61B 17/54; A61B 2017/00734; A61B 2017/00761

(57) ABSTRACT

A non-therapeutic method of abrading skin and a hand-held, cordless skin abrasion device, comprising a handle portion (1) forming part of a housing portion (2), an internal electric drive, a backing pad (3), and an attachable abrasive product (4). The backing pad (3) extends from said housing portion (2), said backing pad (3) is configured to be set in an oscillating motion by said electric drive, and said attachable abrasive product (4) is attached to said backing pad (3) in a mechanical manner, said abrasive product (4) has a laminated structure with a foam core layer (B) and a fabric or film layer (A) which is adhered to an upper side of said foam core layer (B) forming an upper fabric side of said abrasive product, and to a lower side of said foam core layer (B) forming a lower fabric side of said abrasive product (4), and said fabric or film layer (A) is each adhered to said fabric core layer (B) by way of an adhesive layer (D), and said upper fabric side comprises an abrasive layer (C) comprising abrasive particles.

### TECHNICAL FIELD

The present invention relates to abrasive products, and more particularly to skin abrasion devices and non-therapeutic method of abrading skin.

### BACKGROUND

Hyperkeratosis is a skin condition that thickens a person's skin more than usual in certain places. The thickened layer is made of keratin, which is tough, fibrous protein found in fingernails, hair and skin. The body may produce extra keratin because of inflammation, as a protective response to pressure, or because of a genetic condition.

One type of hyperkeratosis is calluses, which are caused by repeated pressure on a spot of the skin. Extra layers of skin grow over the affected area resulting in harder, raised bulges. Calluses are most often found on heels, palms, toes, and knees. Removal of hyperkeratosis part of the skin can also be applied as a prior treatment for psoriasis or actinic keratosis patients.

A natural remedy for removing hyperkeratosis part of skin is by abrasion. A common problem for using abrasion is how to remove thickened skin efficiently but gently without damaging healthy skin in the process.

### BRIEF DESCRIPTION

An object of the present invention is to provide a solution for abovementioned problem.

The invention is based on the idea of providing a skin abrasion device and method, which are stated in independent claims. Preferred embodiments are disclosed in dependent claims.

The device of the present disclosure is a cordless skin abrader system consisting of a soft cushioned coated abrasive product, which can be attached to a powered hand-held machine. With a foam cushioned structure of the coated abrasive product, the risk of scarring the skin during abrasion is minimal in comparison to using conventional sandpaper. In addition, when comparing against other existing methods for removal of hyperkeratotic skin, such as cryotherapy, curette, and laser therapy, the device of the present disclosure is easier to use, more time efficient, and gentler. Thereby, the device of the present disclosure contributes to the medical device market for removal of hyperkeratotic skin and stratum corneum as a painless, effective, easy-to-use and time saving device.

### BRIEF DESCRIPTION OF THE DRAWINGS

In the following the invention will be described in greater detail by means of preferred embodiments with reference to the attached drawings, in which

FIG. 1 shows a schematic view of a skin abrasion device according to an embodiment;

FIG. 2 shows a schematic view of a pad according to an embodiment.

### DETAILED DESCRIPTION

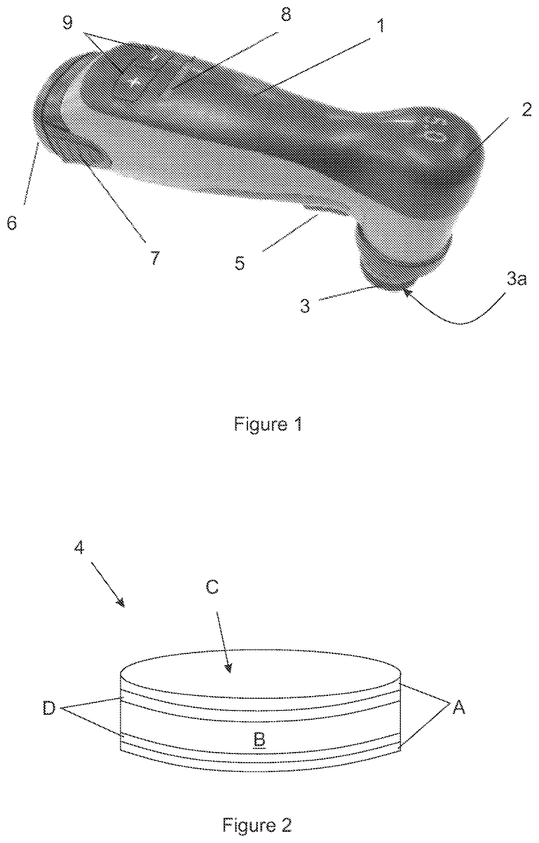

In the following, the invention is described in detail with reference to the drawings. FIG. 1 shows a schematic view of a skin abrasion device without an abrasive product. FIG. 2 shows a schematic view of the abrasive product.

The present invention pertains to a hand-held, cordless skin abrasion device comprising a handle portion 1 forming part of a housing portion 2, an internal electric drive (not shown in Figures), a backing pad 3, and an attachable abrasive product 4. The device may optionally further comprise a dust extractor suitable for the removal of abrasion skin dust, which may be a vacuum system.

The handle portion 1 may be ergonomically formed, wherein its design features a grip portion. The user may align their fingers in a manner where physical effort and discomfort are minimized. The handle portion 1 may have a cylindrical shape without sharp edges or sharp corners. The surface of the grip portion may be smooth or have an anti-slip configuration.

The embodiment illustrated in FIG. 1 comprises a switch 5 for actuating the electric drive. The switch 5 can be a trigger, a lever or a button, for instance. The switch 5 is preferably located underside of the handle portion 1 within easy reach of the user's index finger.

A battery pack 6 may be arranged at an end of the handle portion 1 and preferably partially inside the handle portion 1 and is configured to be removed without the requirement for further tools. The battery pack 6 may be releasably attachable to the handle portion 1 via clamps, for instance. The battery pack 6 may also comprise at least one battery release button 7, preferably two battery release buttons 7 on opposite sides of the battery pack in radial direction. When the user inserts the battery pack 6 inside the handle portion 1, the battery pack 6 may snaplock in place. When the user presses the battery release button 7, the battery pack 6 is released. The battery pack 6 is preferably a rechargeable battery.

The device may also comprise visual means 8 for determining battery life. The visual means 8 may be for instance one or more LED lights indicating the remaining battery charge. For example, green light indicates good battery charge level and red light indicates low battery charge level. In another example, the visual means consists of several LED lights, in which case all lit LED lights indicate full battery and one lit LED light indicates low battery charge level. The visual means 8 may be located on topside of the handle portion 1, where it is easily visible for the user.

The housing portion 2 may be integrally connected to the handle portion 1 in a gun-like manner, wherein the housing portion 2 has a longitudinal axis and the handle portion 1 has a longitudinal axis which forms an angle of 80-120 degrees with the longitudinal axis of the housing portion 2. The angle is more preferably 90 degrees.

The internal electric drive may locate inside the holding portion 2, and can comprise a motor kit connected to the battery pack 6. The motor kit may comprise an internal motor drive and is arranged to produce oscillating and rotational motion via a counterweight, retaining rings, ball bearings and a balance shaft, for instance. However, other type of systems for producing oscillating and rotational motion can be applied. In this context, the term “oscillating motion” may include a repeatable back and forth movement of the backing pad 3. If present, the back-and-forth movement is preferably in axial direction of the backing pad 3, such as a random orbital or rotating motion of the backing pad 3.

The device is arranged to generate predetermined speed of oscillating motion. The device may further comprise speed selector buttons 9. In the accompanying FIG. 1, the speed selector buttons 9 are marked with + and − signs. The user can increase the speed by pressing on the + button and decrease the speed by pressing on the − button.

The backing pad 3 may be attached to the balance shaft with quick-locks and fasteners such as screws, for instance. The backing pad 3 extends from an end of said housing portion 2 and is configured to be set in an oscillating motion by said electric drive. The oscillating motion is 1000 to 15000 rpm (revolutions per minute), preferably 2000 to 12000 rpm, and more preferably 4000 to 8000 rpm by said electric drive. Thanks to the broad spectrum of rpm (1000-15000 rpm) the user is given the opportunity to adjust the speed for a specific kind of surface to be abraded to optimize effectivity and safety.

The device may comprise means for preselecting the rpm of said oscillating motion and/or the length in time of said oscillating motion. In embodiments, the device has an orbital movement, which is gentler than a rotating movement. The gentleness of the orbital movement refers to a uniform speed all over the disc.

In particular, wherein said means comprise means for wireless communication (e.g., using a Bluetooth standard) with a second device and wherein the rpm of said oscillating motion and/or the length in time of said oscillating motion are selected in response to receiving a message defining the rpm of said oscillating motion and/or the length in time of said oscillating motion from said second device via the wireless connection. Said means for wireless communication may be, for instance, a radio transceiver or a receiver. Said second device may be a terminal device or a user equipment, or both. Preselecting a specified maximum running time and/or a constant and/or maximum rpm of said oscillating motion allows for more controlled and higher safety.

The backing pad 3 may comprise an area 3a for the attachable abrasive product 4 to be attached thereon in a mechanical manner. In this context, the term “mechanical manner” refers to a solid, physical type of attachment method which can be manufactured of metal or plastic or composite or mixture thereof. The attachable abrasive product 4 may be attached to said backing pad 3 using a hook and loop system (e.g., Velcro®), in particular wherein the backing pad 3 comprises the hook part of said system, and wherein the abrasive product 4 comprises the loop part of said system positioned at the lower fabric side of said abrasive product 4. In some embodiments, the mechanical manner may include using magnets, vacuum and fasteners, for instance.

In still an embodiment, the abrasive product may comprise a structure as disclosed in WO 2017/174107 A1, which is incorporated herein by reference. In accordance with this embodiment, the abrasive product comprises a knitted cloth on a first side of the flexible abrasive product, grinding agents applied on a second side of the flexible abrasive product facing the first side, and a plurality of loops protruding from the cloth and from the first side, wherein each loop is formed by a pair of bottom-half arcs connected by a protruding head and the loops being arranged in rows extending in the wale direction of the cloth; wherein the bottom-half arcs are interlaced in the cloth, and the protruding heads of the loops are interconnected with one another outside of the cloth so as to form rows of interconnected loops which rows extend in the wale direction of the cloth.

In some embodiments, the abrasive product 4 may be attached to the backing pad 3 in an adhesive manner. In this context, the term “adhesive manner” refers to a non-metallic type of attachment method which comprises a sticky substance. The attachable abrasive product 4 may be attached to said backing pad 3 using an adhesive system, in particular wherein the abrasive product 4 is coated with an adhesive, and the backing pad 3 is adherend. Alternatively, the backing pad 3 may be coated with the adhesive and the abrasive product 4 is the adherend. The adhesive is preferably one of pressure-sensitive adhesives such as elastomer-based compounds. Alternatively, both backing pad 3 and abrasive product 4 may be coated with the adhesive. The adhesive system must allow (i) de- and re-attachment of the abrasive product to the backing pad on the one hand, and (ii) fixation of the abrasive product to the backing pad during the oscillating motion on the other hand.

The backing pad 3 and the abrasive product 4 may each have independently a round shape to which the backing pad 3 and the abrasive product 4 are in contact with each other. The shape is preferably planar and perpendicular to the longitudinal axis of the housing portion 2. The round shape may be in particular a circular shape or an oval shape, and preferably a circular shape. Most preferably the backing pad 3 and the abrasive product 4 may have the same shape. In embodiments, the abrasive product 4 is in the form of an abrasive pad.

The backing pad 3 may have a diameter in the range of 10 to 100 mm, preferably 20 to 77 mm, more preferably 32 to 40 mm, more preferably 32 to 39 mm, more preferably 32 to 38 mm, more preferably 32 to 37 mm, more preferably 32 to 36 mm, more preferably 33 to 35 mm, and most preferably a diameter of about 34 mm at its widest point.

The abrasive product 4 may have a diameter in the range of 10 to 100 mm, preferably 20 to 77 mm, more preferably 32 to 40 mm, preferably 32 to 39 mm, more preferably 32 to 38 mm, more preferably 32 to 37 mm, more preferably 32 to 36 mm, more preferably 33 to 35 mm, and most preferably a diameter of about 34 mm at its widest point.

The diameter of the backing pad 3 may be smaller than the diameter of the abrasive product 4, and wherein the diameter of the backing pad 3 does not differ in more than 4 mm, preferably not more than in 3 mm, more preferably not more than in 2 mm, still more preferably not more than in 1 mm, and most preferably 0.5 mm. Due to the smaller diameter of the backing pad, it is easy to spot if the abrasive product is attached improperly to the backing pad. Generally, the abrasive product should be attached so that the whole backing pad is covered.

The abrasive product 4 may have a thickness of 1 to 10 mm, preferably 4 to 10 mm, more preferably a thickness of 5 to 9 mm, more preferably a thickness of 5 to 8 mm, and even more preferably of 6 to 7 mm.

The abrasive product 4 has a laminated structure with a foam core layer B and a fabric or film layer A (preferably fabric layer A) which is adhered to an upper side of said foam core layer B forming an upper fabric/film side of said abrasive product 4, and to a lower side of said foam core layer B forming a lower fabric side of said abrasive product 4, and said fabric or film layer A (preferably fabric layer A) is each adhered to said foam core layer B by way of an adhesive layer D, and said upper fabric side comprises an abrasive layer C comprising abrasive particles. Thanks to the combination of textile, foam and type of lamination technique, the abrasive particles will bend away from the surface to be abraded when a too high pressure is applied. As a result, the abrasive particles do not cause scarring, but only provide for gentle (but effective) scraping off of the skin surface. As the flexibility of the sanding pad follows the contour of the surface to be abraded, the cushioned foamed sanding pad significantly decrease the risk of scarring the skin in comparison to material containing sharp paper edges as used in conventional abrasive products.

The abrasive product may be a disposable abrasive product. When the abrasive particles have worn out, the abrasive product can be removed and disposed. Likewise, the backing pad may in embodiments be designed to be removed and disposed, when worn out.

The foam core layer B may have a thickness of 1 to 10 mm, preferably 3 to 7 mm, more preferably a thickness of 4 to 6 mm, and most preferably a thickness of about 5 mm.

The foam core layer B may be essentially made of a polymer foam, preferably a synthetic polymer foam, more preferably a polyurethan (PUR) foam, such as a polyether polyurethan (PE-PUR) foam, wherein said foam has a hardness such that it can adapt to the contour of the skin to be abraded. The hotmelt PUR adhesive in layer B may be hardened by drying.

The fabric or film layer A may each independently have a thickness of less than 1 mm, preferably a thickness selected within the range of 0.25 to 0.9 mm, more preferably a thickness selected within the range of 0.4 to 0.8 mm, such as a thickness selected within the range of 0.5 to 0.7 mm.

The fabric layer A may be a woven or non-woven fabric layer made from one or more synthetic polymer fibers, preferably from polyamide fibers.

The abrasive layer C may have a thickness of less than 0.5 mm, in particular a thickness within the range of 0.1 to 0.4 mm, such as a thickness within a range of 0.2 to 0.3 mm.

The abrasive layer C may comprise abrasive particles with a grit number selected within the range of 80 to 4000 according to FEPA, for example 180 to 1000, in particular 360 to 800, such as 500, and/or abrasive particles with an average particle size in diameter of 3 to 201 μm, in particular 20 to 45 μm.

The abrasive particles in said abrasive layer C may be made of silicone carbide (SiC). The abrasive particles may be embedded in said abrasive layer C using a cured resin, e.g. thermally cured resin or radiation cured resin. An example for such a thermally cured resin is phenol formaldehyde resin. Other suitable resins and curing techniques are generally known in the art.

The adhesive layer D may independently each have a thickness of less than 0.5 mm, in particular a thickness within the range of 0.1 to 0.4 mm, such as a thickness within a range of 0.1 to 0.3 mm.

Adhesive techniques may be applied to obtain the adhesive layer D, such as but not limited to flame lamination and dry lamination with thermoplastic adhesive. The adhesive layer D may be a cured hotmelt glue, in particular a cured glue made of a mixture of synthetic plastics, preferably a mixture of synthetic plastics based on polyurethane. The adhesive layer D may alternatively be a thermoplastic adhesive web of a solid material with a nonwoven structure.

A skin abrasion kit may comprise the skin abrasion device and at least one additional abrasive product 4 as replacement. The kit may comprise at least two different abrasive products 4, which differ in their respective grit number and/or average particle size of the abrasive particles, preferably wherein the abrasive particles have grit sizes selected from 180, 360, 500 and 800 according to FEPA.

The kit may further comprise a replacement battery for the device, in particular a rechargeable replacement battery for the device and a charging device for charging the battery of said device.

The kit may further comprise a dust extractor suitable for the removal of abrasion skin dust.

The kit may further comprise user protection equipment, in particular a protective mask and/or gloves.

In addition, the kit may further comprise instructions for use of the device and/or the abrasive product in accordance with the following disclosure concerning methods which apply the device of the present disclosure.

Generally, the device can be used in both non-therapeutic methods and therapeutic methods, dependent on what type of skin is abraded. For example, in non-therapeutic methods thickened skin may be removed for cosmetic reasons, e.g. from feet such as to obtain smooth-skinned feet. In this type of use, the device may be used in a similar way than a foot file or corneal file. Likewise, the device may also be used for therapeutic purposes on pathological changes of the skin, as known to the person skilled in the art.

In accordance with the foregoing, the present disclosure provides a method of abrading skin from at least one subject, in particular a non-therapeutic method for abrading skin from at least one subject, comprising the steps of
(a) providing a skin abrasion device as described herein above comprising a first attachable abrasive product with a selected first particle size of the abrasive particles;
(b) conducting a first abrading step, wherein skin is abraded from a subject using the skin abrasion device of step (a);
(c) removing said first attachable abrasive product from said skin abrasion device, and attaching a second attachable abrasive product with a selected second particle size of the abrasive particles; and
(d) conducting a second abrading step, wherein skin is abraded from a subject using the skin abrasion device resulting from step (c).

In preferred embodiments, the skin is human skin. The skin to be abraded is usually cleaned prior to abrasion. Hair removal is not necessary as the hair will gently be removed during abrasion. In each abrading step, the powered device is turned on and the oscillating movements contribute to abrasion of the hyperkeratotic skin as the device is applied parallel onto the skin. The abrasion of skin should be stopped when the hyperkeratotic part of skin has been removed. Examination of completed treatment is mainly conducted by eye-hand examination, but can secondly conducted (e.g. during therapeutic treatments) by examination with a dermatoscope. Remaining skin dust may be removed from the abraded skin with a cleansing wipe.

Method steps (c) and (d) may be repeated at least one more time, such as twice or three times. In a particular embodiment, the subject in step (b) and step (d) is the same subject. In this case the second particle size is usually the same or smaller than the first particle size. Indeed, most often the second particle size is smaller than the first particle size. A suitable abrasive product is often chosen with a grit number between 360 and 1000, according to hardness of the hyperkeratotic skin to be abraded. Typically the harder and the thicker the skin, the lower the grit number. As disclosed herein above, the abrasive particles may have at least grit sizes selected from 80-4000 according to FEPA. Grit sizes of 500 and finer are of largest interest for removal of hyperkeratotic skin on sensitive skin areas, whereas grit sizes of 360 and coarser are of largest interest for podiatry. A new abrasive product may also be used when another part of the body is to be abraded. The abrasive product is discarded after use.

In embodiments, the method further comprises after step (a) and prior to step (b), and/or after step (b) and prior to step (d), an additional step of preselecting the rpm of the oscillating motion of said device and/or the length in time of said oscillating motion of said device. In embodiments, the rpm and/or the time of said oscillating motion is selected via a wireless connection using a second device. The wireless connection may use, for example, a Bluetooth standard. In accordance with the device disclosed herein above, the rpm of said oscillating motion may be selected within the range of 1000 to 15000 rpm, preferably 2000 to 12000 rpm, and more preferably 4000 to 8000 rpm.

If the method is applied in a therapeutic method, the method removes hyperkeratotic skin from said subject, which has a skin disease treatable by skin abrasion. For example, said subject may have been diagnosed of having psoriasis, or said subject may suffer from a pathogenic skin callus. In embodiments, the method removes hyperkeratotic skin from said subject, wherein said subject is in need of medical chiropody (medical foot care). In case the device is used in a therapeutic method, the device may be used in steps (b) and/or (d) for percutaneous delivery of an active substance, such as for percutaneous delivery of an active substance in the treatment of actinic keratosis prior to photodynamic therapy (PDT).

Generally, the abrasive product should be attached so that the whole backing pad is covered. As noted herein above, the dimensions of the backing pad and the abrasive product allow to easily spot if the abrasive product is correctly attached. If the abrasive product may not be correctly attached, the device of the present disclosure advantageously allows to remove the pad and re-attach same properly. As noted herein above, this may be achieved using, for example, a hook and loop system for attaching the abrasive product to the backing pad.

In embodiments, it is recommendable to apply user protection equipment, such as gloves, a protective mask, and/or a dust extractor in the operating room (in case the device is not equipped with a dust extractor). Suitable dust extractors are medical vacuum system for removal of the abrasion skin dust.

After use, the powered hand-held device may be cleaned using non-alcoholic agent detergent wipes, and/or the battery is charged.

Finally, the present disclosure also provides a method of producing an abrasive product according to the present disclosure, wherein the method comprises the steps of
(a) laminating a fabric or film layer (A) to an upper side of a foam core layer (B) forming an upper fabric side, and to a lower side of said foam core layer (B) forming a lower fabric side, wherein said fabric layer (A) is each adhered to said fabric core layer (B) by way of an adhesive layer (D), wherein said adhesive layer (D) is a hotmelt glue,
(b) hardening the hotmelt glue by drying;
(c) embedding abrasive particles into a resin, and applying the embedded particles to said upper fabric side to form an abrasive layer (C) comprising abrasive particles;
(d) curing the resin of said abrasive layer C by heating; and
(e) cutting out the abrasive product by punching.

The laminating step (a) may comprise flame lamination or dry lamination with thermoplastic adhesive.

The hardening step (b) may comprise drying the adhesive layer in room temperature for few seconds to 10 minutes, preferably 5-30 seconds.

Embedding abrasive particles in step (c) can be implemented with any known process, such as but not limited to coating. The resin is preferably a solid resin, such as phenol formaldehyde, epoxy, polyimide, alkyd, non-saturated polyester and polyamide resins.

The curing step (d) may comprise heating the resin in 100-200° C. for 12-72 hours, preferably 24 hours. In some embodiments, curing can alternatively be initiated by radiation, electron beams, or chemical additives.

The cutting step (e) may comprise punching the predetermined shape of the abrasive product from a blank by shearing force using a punch and die.

Before the laminating step (a) the layers may be strewed electrostatically to prevent forming wrinkles on the abrasive product.

In embodiments, the fabric forming the lower fabric side comprises one part of a hook and loop system for attaching said pad to a dermabrasive device, in particular wherein the lower fabric side comprises the loop part of said system.

In further embodiments, the punching die used in step (e) has a round shape, in particular a circular shape or an oval shape, preferably a circular shape. The punching die used in step (e) may have an inner diameter in the range of 10 to 100 mm, preferably 20 to 77 mm, more preferably 32 to 40 mm, preferably 32 to 39 mm, more preferably 32 to 38 mm, more preferably 32 to 37 mm, more preferably 32 to 36 mm, more preferably 33 to 35 mm, and most preferably a diameter of about 34 mm at its widest point.

The abrasive product obtained in step (e) may have a thickness of 1 to 10 mm, preferably 4 to 10 mm, preferably a thickness of 5 to 9 mm, more preferably a thickness of 5 to 8 mm, and even more preferably of 6 to 7 mm.

In embodiments, said foam core layer (B) has a thickness of 1 to 10 mm, preferably 3 to 7 mm, preferably a thickness of 4 to 6 mm, and most preferably a thickness of about 5 mm. In embodiments, said foam core layer (B) is essentially made of a polymer foam, preferably a synthetic polymer foam, more preferably a polyurethan foam, such as a polyether polyurethan foam; wherein said foam has a hardness such that it allows adapting the pad to the contour of the skin to be abraded.

The fabric or film layer (A) may each independently have a thickness of less than 1 mm, preferably a thickness selected within the range of 0.25 to 0.9 mm, more preferably a thickness selected within the range of 0.4 to 0.8 mm, such as a thickness selected within the range of 0.5 to 0.7 mm. In embodiments, said fabric layer (A) is a woven or non-woven fabric layer made from one or more synthetic polymer fibers, preferably from polyamide fibers.

The abrasive layer (C) may have a thickness of less than 0.5 mm, in particular a thickness within the range of 0.1 to 0.4 mm, such as a thickness within a range of 0.2 to 0.3 mm. In embodiments, said abrasive particles may have a grit number selected within the range of 80 to 4000 according to FEPA, for example 180 to 1000, in particular 360 to 800; and/or have an average particle size in diameter of 20 to 45 μm. The abrasive particles may be made of any suitable material. In some embodiments, said abrasive particles are made of silicone carbide. The abrasive particles may be fixed to the abrasive layer by way of any suitable means, and/or as described herein above.

The adhesive layer (D) may independently have each a thickness of less than 0.5 mm, in particular a thickness within the range of 0.1 to 0.4 mm, such as a thickness within a range of 0.1 to 0.3 mm. In embodiments, said hotmelt glue of said adhesive layer (D) is a glue made of a mixture of synthetic plastics, preferably a mixture of synthetic plastics based on polyurethane.

1. A hand-held, cordless skin abrasion device, comprising
a housing portion,
an internal electric drive,
a backing pad, and
an attachable abrasive product,
and optionally further comprising a dust extractor suitable for the removal of abrasion skin dust,
wherein
said backing pad extends from said housing portion,
said backing pad is configured to be set in an oscillating and/or random orbital or rotating motion by said electric drive, and
said attachable abrasive product is attached to said backing pad in a mechanical manner,
said abrasive product has a laminated structure with a foam core layer and a fabric or film layer which is adhered to an upper side of said foam core layer forming an upper fabric or film side of said abrasive product, and to a lower side of said foam core layer forming a lower fabric or film side of said abrasive product, and
said fabric or film layer is each adhered to said foam core layer by way of an adhesive layer, and
said upper fabric or film side comprises an abrasive layer comprising abrasive particles.

2. The device of claim 1, wherein said device is driven by a battery, in particular wherein the battery is a battery configured to be removed without the requirement for further tools, in particular wherein the battery is a rechargeable battery.

3. The device of claim 1, wherein the handle portion is ergonomically formed, and/or wherein the handle portion and the housing portion are arranged in a gun-like manner.

4. The device of claim 1, wherein said backing pad is configured to be set in an oscillating motion of 1000 to 15000 rpm, preferably 2000 to 12000 rpm, and more preferably 4000 to 8000 rpm by said electric drive.

5. The device of claim 1, wherein said device comprises means for preselecting the rpm of said oscillating motion and/or the length in time of said oscillating motion, in particular, wherein said means comprise means for wireless communication with a second device and wherein the rpm of said oscillating motion and/or the length in time of said oscillating motion are selected in response to receiving a message defining the rpm of said oscillating motion and/or the length in time of said oscillating motion from said second device via the wireless connection.

6. The device of claim 1, wherein said attachable abrasive product is attached to said backing pad using a hook and loop system;
in particular wherein the backing pad comprises the hook part of said system, and wherein the abrasive product comprises the loop part of said system positioned at the lower fabric side of said abrasive product;
optionally wherein the abrasive product is a disposable abrasive product.

7. The device of claim 1, wherein the backing pad and the abrasive product each have independently a round shape, in particular a circular shape or an oval shape, preferably a circular shape, most preferably the backing pad and the abrasive product have the same shape;
in particular wherein the backing pad has a diameter in the range of 10 to 100 mm, preferably 20 to 77 mm, more preferably 32 to 40 mm, preferably 32 to 39 mm, more preferably 32 to 38 mm, more preferably 32 to 37 mm, more preferably 32 to 36 mm, more preferably 33 to 35 mm, and most preferably a diameter of about 34 mm at its widest point, and/or wherein the abrasive product has a diameter in the range of 32 to 40 mm, preferably 32 to 39 mm, more preferably 32 to 38 mm, more preferably 32 to 37 mm, more preferably 32 to 36 mm, more preferably 33 to 35 mm, and most preferably a diameter of about 34 mm at its widest point;
in particular wherein the diameter of the backing pad is smaller than the diameter of the abrasive product, and wherein the diameter of the backing pad does not differ in more than 4 mm, preferably not more than in 3 mm, more preferably not more than in 2 mm, still more preferably not more than in 1 mm, and most preferably 0.5 mm.

8. The device of claim 1, wherein the abrasive product has a thickness of 1 to 10 mm, preferably 4 to 10 mm, preferably a thickness of 5 to 9 mm, more preferably a thickness of 5 to 8 mm, and even more preferably of 6 to 7 mm.

9. The device of claim 1, wherein said foam core layer (B) has a thickness of 1 to 10 mm, preferably 3 to 7 mm, preferably a thickness of 4 to 6 mm, and most preferably a thickness of about 5 mm;
and/or wherein said foam core layer is essentially made of a polymer foam, preferably a synthetic polymer foam, more preferably a polyurethan foam, such as a polyether polyurethan foam; wherein said foam has a hardness such that it can adapt to the contour of the skin to be abraded.

10. The device of claim 1, wherein said fabric or film layer each independently has a thickness of less than 1 mm, preferably a thickness selected within the range of 0.25 to 0.9 mm, more preferably a thickness selected within the range of 0.4 to 0.8 mm, such as a thickness selected within the range of 0.5 to 0.7 mm;
and/or wherein said fabric layer is a woven or non-woven fabric layer made from one or more synthetic polymer fibers, preferably from polyamide fibers.

11. The device of claim 1, wherein said abrasive layer has a thickness of less than 0.5 mm, in particular a thickness within the range of 0.1 to 0.4 mm, such as a thickness within a range of 0.2 to 0.3 mm;
and/or wherein said abrasive layer comprises abrasive particles with a grit number selected within the range of 80 to 4000 according to FEPA, in particular 180 to 1000, in particular 360 to 800, such as 500; and/or abrasive particles with an average particle size in diameter of 3 to 201 μm, in particular 20 to 45 μm;
optionally wherein said abrasive particles in said abrasive layer are made of silicone carbide.

12. The device of claim 1, wherein said abrasive particles are embedded in said abrasive layer using a cured resin, in particular thermally cured resin or radiation cured resin.

13. The device of claim 1, wherein said adhesive layer has independently each a thickness of less than 0.5 mm, in particular a thickness within the range of 0.1 to 0.4 mm, such as a thickness within a range of 0.1 to 0.3 mm;
and/or wherein said adhesive layer is a cured hotmelt glue, in particular a cured glue made of a mixture of synthetic plastics, preferably a mixture of synthetic plastics based on polyurethane.

14. A non-therapeutic method of abrading skin from at least one subject,
comprising the steps of
(a) providing a skin abrasion device as defined in claim 1, comprising a first attachable abrasive product with a selected first particle size of the abrasive particles;
(b) conducting a first abrading step, wherein skin is abraded from a subject using the skin abrasion device of step (a);
(c) removing said first attachable abrasive product from said skin abrasion device, and attaching a second attachable abrasive product with a selected second particle size of the abrasive particles; and
(d) conducting a second abrading step, wherein skin is abraded from a subject using the skin abrasion device resulting from step (c);
optionally wherein method steps (c) and (d) are repeated at least one more time.

15. The method of claim 14, wherein the subject in step (b) and step (d) is the same subject;
in particular wherein the second particle size is the same or smaller than the first particle size, preferably wherein the second particle size is smaller than the first particle size.

16. The method of claim 14, wherein the method further comprises after step (a) and prior to step (b), and/or after step (b) and prior to step (d), preselecting the rpm of the oscillating motion of said device and/or the length in time of said oscillating motion of said device; optionally wherein the rpm and/or the time of said oscillating motion is selected via a wireless connection using a second device, in particular wherein said wireless connection uses a Bluetooth standard;
in particular wherein the rpm of said oscillating motion is elected within the range of 1000 to 15000 rpm, preferably 2000 to 12000 rpm, and more preferably 4000 to 8000 rpm.
